(19) United States
(12) Patent Application Publication
(10) Pub. No.: US 2011/0117571 A1
(43) Pub. Date: May 19, 2011

(54) Transporter assay

(75) Inventors: Heini Frang, Lieto (FI); Jari Hovinen, Raisio (FI); Veli-Matti Mukkala, Kaarina (FI); Pertti Hurskainen, Piispanristi (FI); Ilkka Hemmilä, Sauvo (FI)

(73) Assignee: WALLAC OY, Turku (FI)

(21) Appl. No.: 12/743,236

(22) PCT Filed: Nov. 27, 2008
(86) PCT No.: PCT/FI08/50696

§ 371 Date: May 17, 2010

Related U.S. Application Data
(60) Provisional application No. 61/013,628, filed on Dec. 13, 2007.

(30) Foreign Application Priority Data
Nov. 30, 2007 (FI) 20070926

(51) Int. Cl. G01N 33/53; G01N 33/50

(52) U.S. Cl. 435/7.1; 436/501; 436/86; 436/94

(57) ABSTRACT

This invention concerns a non-radioactive homogenous proximity assay for cellular transport system. The assay format disclosed here takes advantageous of the fact that ABC transporters have two similar ATP binding sites, and thus allowing two ATP molecules to bind simultaneously to these adjacent sites.

### FIELD

The technology described herein relates to an assay of measuring active molecular transport system out of the cells by ATP-binding cassette transporters.

### BACKGROUND

Toxic side effects of drug have become the major obstacle in developing new block-buster pharmaceuticals. To improve the efficacy and in particular the safety of novel drug candidates, one has to assess the toxic effects of novel lead compounds in early phase in high-throughput mode. Toxicity relates also to specific transporter systems, which specifically pump small molecular compounds out from cells using ATP as energy source. In terms of adverse effect, the vital organs, such as liver, brain, heart muscles, has to be addressed.

ABC (ATP-binding cassette) transporters are one of the largest and most ancient (conserved) families of transporters present from prokaryotic organism to humans. These ABC transporters are transmembrane proteins that export structurally diverse hydrophobic compounds from the cell driven by ATP hydrolysis. One of the most studied ABC transporter is the P-glycoprotein (PGP) which is e.g. known to play central role in the absorption and distribution of drugs in many organisms and organs. PGP forms a major component of the blood-brain barrier. Its role is also to prevent the entry of potentially toxic compounds from the gut into the blood and protect sensitive internal organs. On the other hand, PGP and the other ABC transporters in general can also reduce the oral bioavailability of the therapeutic drug and the targeting of such drugs to the brain tissue, limiting the efficacy of treatment.

Many of the commonly used drugs are PGP substrates. Compounds that interact with PGP can function as stimulators or inhibitors of its ATPase activity. Over expression of ABC transporters has been also linked to efflux of chemotherapeutic drugs used for cancer treatment, sometimes leading to multidrug resistance problem. ABC transporters also play an important role in certain adverse drug-drug interactions.

ATP hydrolysis by transporters takes place at the two nucleotide binding (NB) domains located on the cytoplasmic face of the protein. ABC transporter consist of two homologous halves, each with six transmembrane (TM) segments and a cytosolic NB domain. The drug-binding site is formed by the TM regions of both halves of PGP. Substrates gain entry to this site from within the membrane. Nucleotide binding causes repacking of the TM regions of PGP, thereby opening the central pore to allow access of hydrophobic drugs directly form the lipid bilayer, leading to the proposal that ATP binding, rather than hydrolysis, drives the conformational changes associated with transport. First there is the catalytic cycle whereby ATP is hydrolyzed; this comprises ATP binding, formation of a putative nucleotide sandwich dimer, hydrolysis of ATP, dissociation of N and dissociation of ADP. The energy derived from this cycle is coupled to substrate movement across the membrane.

There are methods to measure specific transporters, none of which, however, is very efficient and suitable for high-throughput application.

The traditional monolayer efflux assay is regarded as the standard for identifying PGP substrates because this assay measures efflux in the most direct manner. However, monolayer assays are labour-intensive due to need of constant cell culturing and thus this assay is not amenable to automation.

ABC transporters can be also studied in membrane vesicles prepared from cells over expressing the wanted transporter. Inside-out membrane vesicles are good tools for calcein AM fluorescence based method monitoring the transporter efflux. Calcein AM is a substrate for the ABC transporters and it is intracellularly converted to a fluorescent product. However, this assay is not designed to distinguish PGP substrates from inhibitors, and do not directly measure transport. The method as such can be automated.

PerkinElmer has developed a non-radioactive heterogeneous GTP binding assay to monitor activation of G protein-coupled receptors. The assay exploits the unique fluorescence properties of lanthanide chelates. The assay is based on a GTP analogue labelled with a europium chelate and membrane fragments, all bound to a filtration plate. The labelled GTP derivative has an enhanced stability towards enzymatic hydrolysis. The same assay format could be adapted to the corresponding ABC transported assay by substituting the labelled GTP derivative with the corresponding ATP analogue.

The major drawback of heterogeneous assays is the requirement for extensive washings and prolonged incubations making their automation demanding.

Solvo Company has developed a homogeneous assay monitoring colorimetrically the release of inorganic phosphate by ATP hydrolysis. Instability of the signal makes the assay difficult to automate and to perform in high-throughput format although this assay is readily automated.

### OBJECTS AND SUMMARY OF THE INVENTION

The main objective of the present invention is to provide an easily automated, high-throughput proximity assay for cellular transport system.

The homogenous assay format disclosed here takes advantageous of the fact that ABC transporters have two similar ATP binding sites. Accordingly, two ATP molecules are able to bind simultaneously to these adjacent NB sites.

According to one aspect of this invention, in addition to ATP derivatives, the assay can utilize other binding molecules binding to NB sites or adjacent to NB sites. Such molecules can be used as carrier of one partner of proximity assay, and such a molecule may comprise antibodies, oligopeptides, polypeptides, oligonucleotides polynucleotises, lectins or other natural or artificial polymers either mimicking ATP binding or recognizing adjacent motifs of NB sites.

According to one aspect this invention the signal detection is based on various forms of proximity assays. Examples of proximity assays include fluorescence energy transfer, fluorescence energy quenching, energy transfer between upconverted particles and fluorescent acceptors, fluorescence cross-correlation, luminescent oxygen channelling, and enzyme fragment complex formation upon proximity.

According to one aspect this invention concerns an assay where the energy transfer signal is detected between two labelled ATP derivatives when bound to ATP-binding cassette, wherein one of the ATP derivatives is labelled with an energy donor and the other one with an energy acceptor.

Thus, according to one aspect this invention concerns an assay wherein the energy acceptors are labelled ATP conjugates comprising a fluorometric or luminometric label.

According to another aspect this invention concerns an assay wherein the energy donors are labelled ATP conjugates comprising a fluorometric or luminometric label.

According to another aspect this invention concerns an assay wherein the energy acceptors are antibodies, oligopeptides, polypeptides, oligonucleotides polynucleotides, lectins or other natural of artificial polymers either mimicking ATP binding or recognizing adjacent motifs of NB sites labelled with fluorometric or luminometric label.

According to another aspect this invention concerns an assay wherein the energy donors are anti-transporter antibodies, lectins, polypeptides, polynucleotides, oligonucleotides, oligopeptides, anti-tag antibodies or other natural or artificial polymers either mimicking ATP binding or recognizing adjacent motifs of NB sites labelled with fluorometric or luminometric label.

According to another aspect this invention concerns an assay wherein the labelled ATP derivatives have an enhanced stability towards nucleases.

According to one aspect, the invention is based on a novel method to develop binding-domain compatible, non-hydrolyzable ATP conjugates containing a suitable label moiety enabling the measurement of transporter activation easily and quantitatively. The labeled ATPs bind to transporter binding domain when the transporter is activated with a drug or other molecule under examination, and since the ATP derivatives are not hydrolyzed, they allow the quantitation of activated transporter.

### DETAILED DESCRIPTION OF THE INVENTION

As defined herein, “a transporter binding molecule” refers to a labelled ATP derivative, anti-transporter antibody, lectin, polypeptide, polynucleotide, oligonucleotide, oligopeptide, anti-tag antibody and other molecule capable in binding ATP binding sites and other natural and artificial polymers either mimicking ATP binding or recognizing adjacent motifs of NB sites

As defined herein, “ABC transporter” is a family of membrane transport proteins that use the energy of ATP hydrolysis to transport various molecules across the membrane.

As defined herein “ATP-binding cassette transporters (ABC-transporter)” are members of a superfamily with representatives in all extant phyla from prokaryotes to humans. These are transmembrane proteins that function in the transport of a wide variety of substrates across extra- and intracellular membranes, including metabolic products, lipids and sterols, and drugs. Proteins are classified as ABC transporters based on the sequence and organization of their ATP-binding domain(s), also known as nucleotide-binding (NB) domains. ABC transporters are involved in tumour resistance, cystic fibrosis, bacterial multidrug resistance and a range of other inherited human diseases.

As defined herein, “a stable ATP derivative” refers to a labelled ATP derivative with enhanced stability towards nucleases.

The invention disclosed herein comprises a homogenous non-radioactive proximity assay for ABC transporter activity wherein detection of the ABC transporter activity is based on a signal between two labeled ABC transporter binding molecules.

“The sites adjacent to ATP binding domains can be any suitable binding sites on the same transporter complex, which together with one reagent bound to ATP binding domain, allow direct monitoring of the transporter activation by energy transfer.”

“Proximity assay” means a situation, wherein labels through binding reaction (for example energy donoring chelate label and energy accepting organic fluorescence label) come so close to each other that non radiating (Förster) energy transfer can occur. This distance is in general less than 20 nm.

According to one embodiment, the labelled ABC transporter binding molecules are ATP derivatives, anti-transporter antibodies, lectins, polypeptides, polynucleotides, oligonucleotides, oligopeptides, or anti-tag antibodies. According to a preferable embodiment, the ABC transporter binding molecules are ATP derivatives.

According to another embodiment the signal detection is based on fluorescence energy transfer, fluorescence energy quenching, energy transfer between upconverted particles and fluorescent acceptors, fluorescence cross-correlation, luminescent oxygen channelling, and enzyme fragment complex formation upon proximity. In particular embodiment the signal detection is based on time-resolved fluorescence energy transfer or time-resolved fluorescence energy quenching.

According to another embodiment the ABC transporter binding molecules are labelled with luminescent lanthanide(III) chelates, quantum dots, nanobeads, upconverting phosphors or organic dyes. According to a preferable embodiment, the organic dye is selected from alexa dyes, cyanine dyes, dabcyl, dancyl, fluorescein, rhodamine, TAMRA and bodiby.

According to another embodiment one of the ATP derivatives is labelled with a luminescent lanthanide(III) chelate and one of the ATP derivatives is labelled with an organic dye. The lanthanide(III) chelate acts as a energy donor and the organic dye acts as an energy acceptor.

In a particular embodiment two ATP molecules bind to transporter in its activation. Because the binding domains are situated near each other, transporter activation bring the two labels in proximity allowing energy transfer between them in active complex when used in suitable concentrations.

It is desirable that the ATP derivatives have enhanced stability towards nucleases. This can be achieved by substituting one or more of the oxygen atoms of the triphosphate moiety by carbon, sulphur or nitrogen. Representative structures are ATPaS, ATPyS, ApCpp, AppCp, and AppNHp. These modified ATP derivatives are commercially available.

The label can be attached to the ATP molecule either directly or via a linker arm. Suitable sites are for labelling are C8 of the adenine moiety, O2′— or O3′— of the sugar moiety and γ-phosphate of the triphosphate moiety. Labelling at γ-phosphate also enhances the nuclease resistance of the said triphosphate.

In a particular embodiment the labelled ATP derivative is selected from a group consisting of

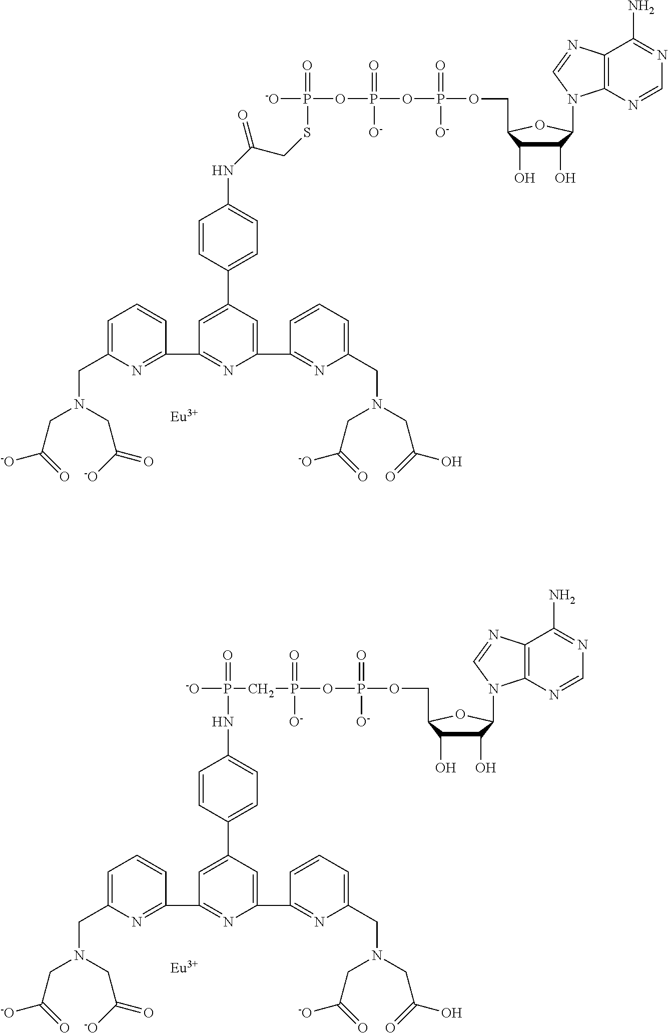

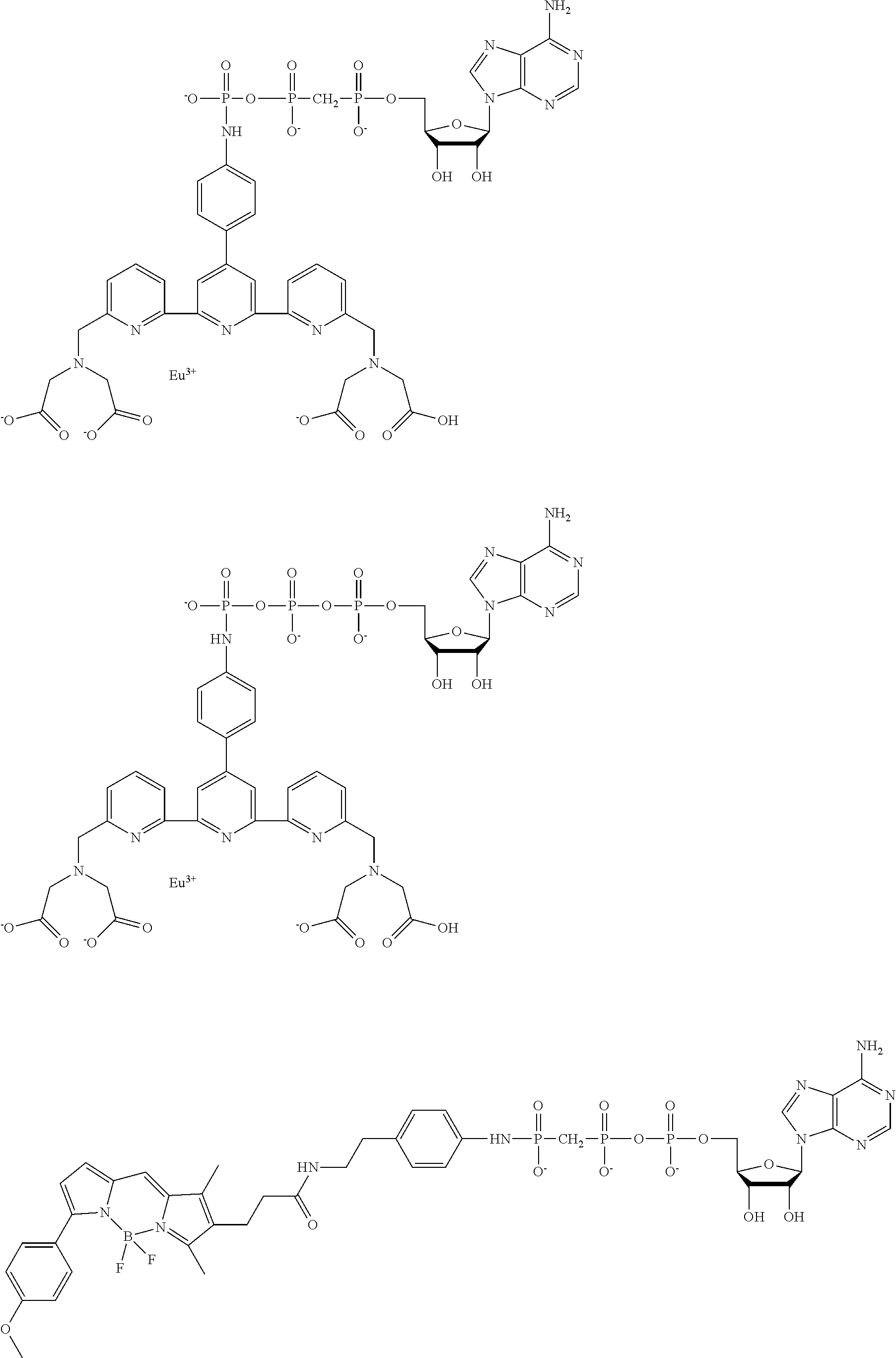

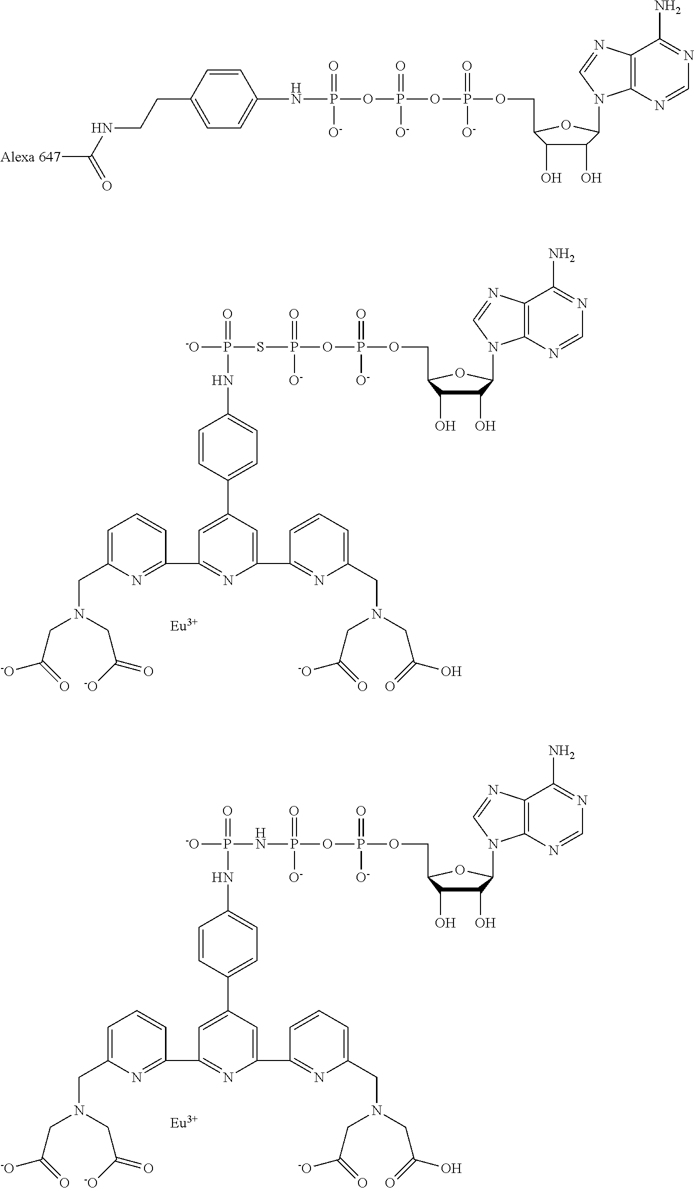

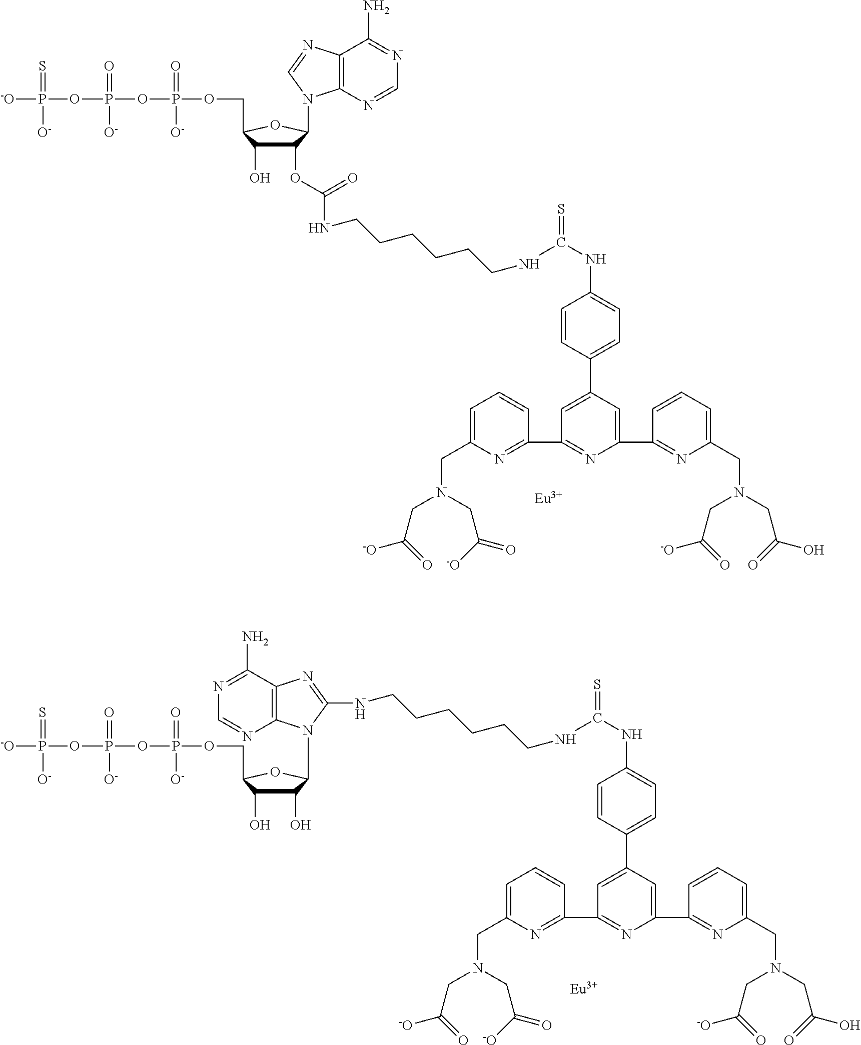

### BRIEF DESCRIPTION OF DRAWINGS

FIG. 1. A dose-response curve of PGP transporter using verapamil as stimulating drug. 10 nM Eu-labelled ATP (donor; Example 1) and 10 nM Alexa647-labeled ATP (acceptor; Example 5) were used to detect the transporter activity. Energy transfer was measured in the plate reader after 2 h incubation. 2 μg of Sf9 membranes were used/well.

The invention will be illuminated by the following non-restrictive examples.

### EXAMPLES

The invention is further elucidated by the following examples.

General. Electrospray mass spectra were recorded on an Applied Biosystems Mariner ESI-TOF instrument. HPLC purifications were performed using a Shimazu LC 10 AT instrument equipped with a diode array detector, a fraction collector and a reversed phase column (LiChrocart 125-3 Purospher RP-18e 5 μm). Mobile phase: (Buffer A): 0.02 M triethylammonium acetate (pH 7.0); (Buffer B): A in 50% (v/v) acetonitrile. Gradient: from 0 to 1 min 95% A, from 1 to 31 min from 95% A to 100% B. Flow rate was 0.6 mL min.⁻¹

### Example 1

### Synthesis of the derivative between adenosine 5′[γ-thio]triphosphate and {2,2′,2′,2″-{[4′-(4′″-iodoacetamidophenyl)-2,2′:6′,2″-terpyridine-6,6″-diyl]bis(methylenenitrilo)}tetrakis(acetate)}europium(III)

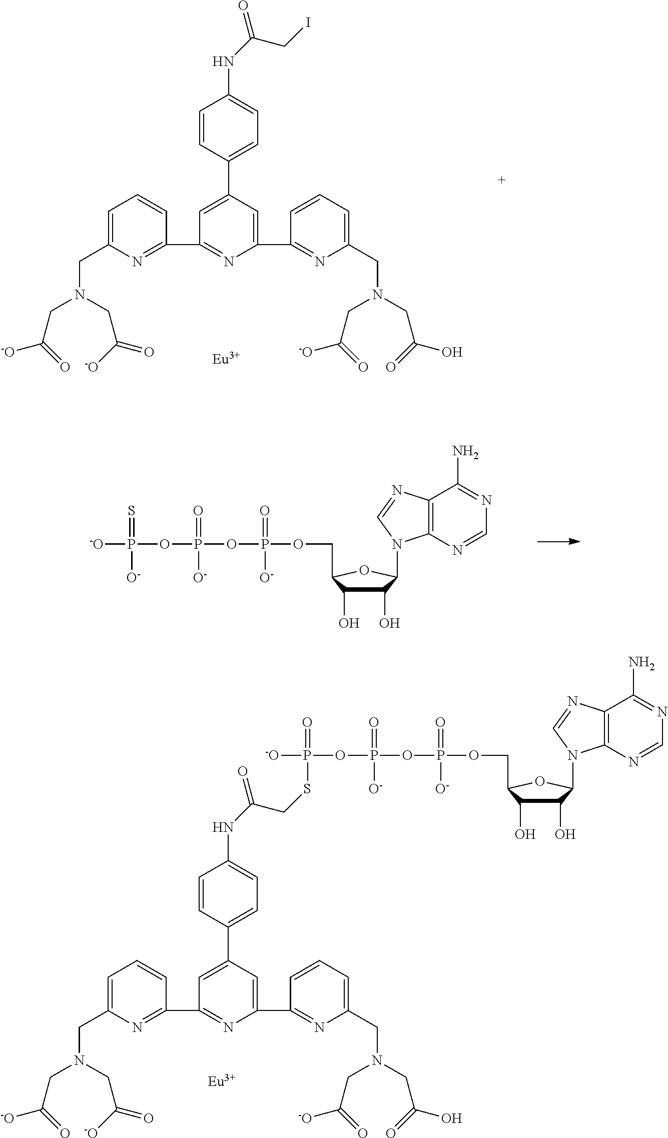

Adenosine 5′-[γ-thio]triphosphate tetralithium salt (1.2 mg) and {2,2′,2″,2′″-{[4′-(4′″-iodoacetamidophenyl)-2,2′:6′,2″-terpyridine-6,6″-diyl]bis(methylene-nitrilo)}tetrakis(acetate)}europium(III) (4.2 mg) were dissolved in water and stirred for 2.5 hours at room temperature. The product was purified with HPLC and was analyzed with ESI-TOF mass spectrometry.

### Example 2

### Amide of adenosine 5′[β,γ-methylene]triphosphate with {2,2′,2″,2″-{[4′-(4′″-aminophenyl)-2,2′:6′,2″-terpyridine-6,6″-diyl]bis(methylenenitrilo)}-tetrakis(acetate)}europium(III)

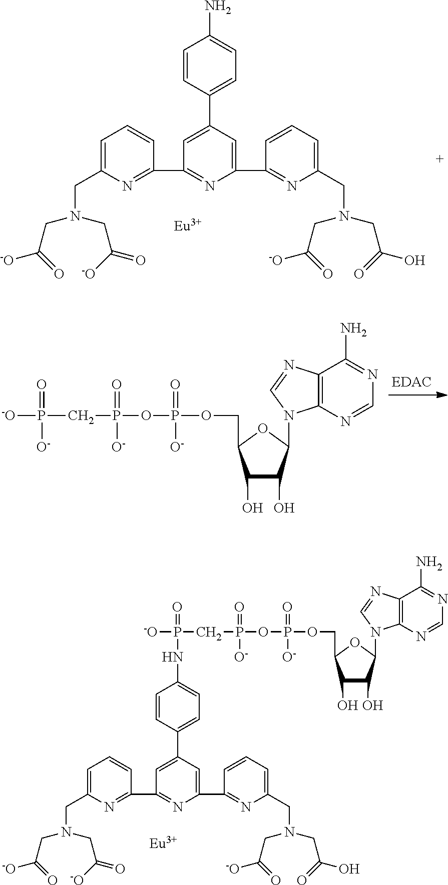

Adenosine 5′[β,γ-methylene]triphosphate (2.9 mg) and {2,2′,2″,2′″-{[4′-(4′″-aminophenyl)-2,2′:6′,2″-terpyridine-6,6″-diyl]bis(methylenenitrilo)}tetrakis-(acetate)}europium(III) (3.4 mg) were dissolved in 0.5 M MES buffer, pH 5.5 (100 μL). EDAC (3.0 mg) was added and the reaction mixture was stirred overnight at RT. The product was precipitated with acetone. The precipitation was washed with acetone. The product was purified with HPLC and was analyzed with ESI-TOF mass spectrometry.

### Example 3

### Amide of adenosine 5′[α,β-methylene]triphosphate with {2,2′,2″,2′″-{[4′-(4′″-aminophenyl)-2,2′:6′,2″-terpyridine-6,6″-diyl]bis(methylenenitrilo)}-tetrakis(acetate)}europium(III)

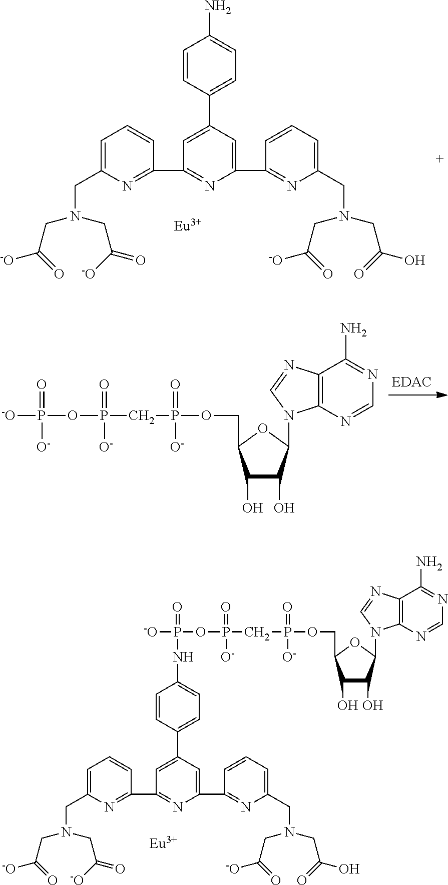

The title compound was synthesized analogously with Example 2 using adenosine 5′-[α,β-methylene]triphosphate as a starting material.

### Example 4

### Amide of adenosine 5′-[β,γ-methylene]triphosphate with BODIPY-TMR

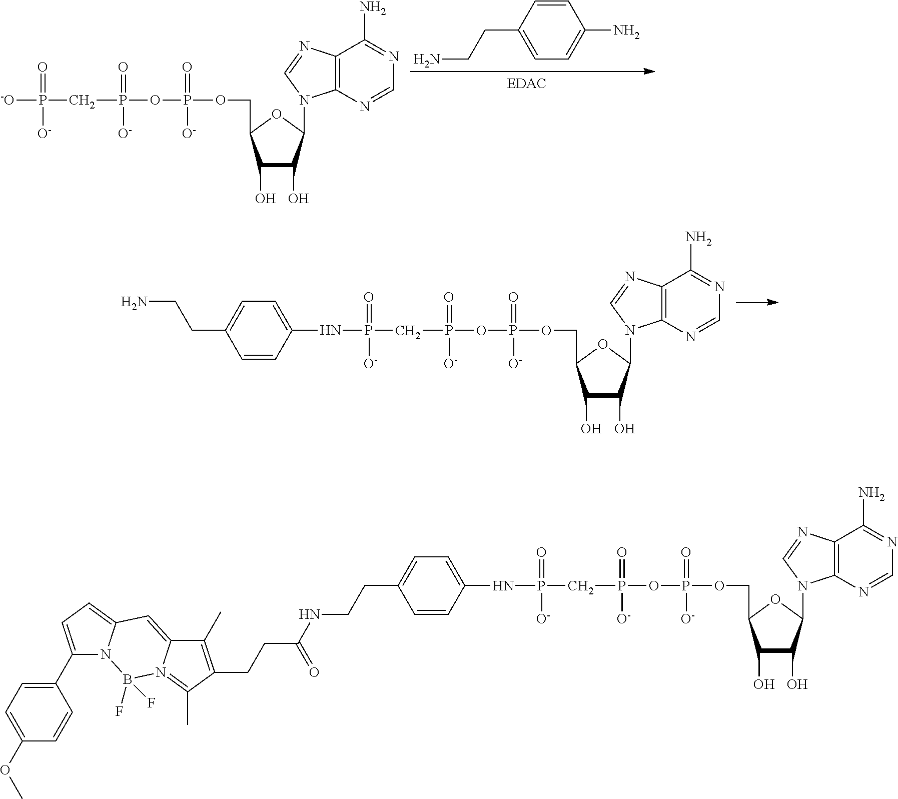

Adenosine 5′[β,γ-methylene]triphosphate (2.0 mg), 2-(4-aminophenyl)ethylamine (10.4 mg) and EDAC (5.3 mg) were dissolved in MES buffer (200 μL, 0.5 M, pH 5.0), and the reaction was allowed to proceed overnight at room temperature. The product was precipitated with acetone, and the precipitation was washed with the same solvent. The precipitate was dissolved in a mixture of a carbonate buffer (500 μL, 0.1M; pH 8.6) and dioxane (500 μL). BODIPY-TMR NHS (0.7 mg) was added, and the mixture was stirred overnight. The product was precipitated with acetone and was washed with the same solvent. The product was purified with HPLC was analyzed with ESI-TOF mass spectrometry.

### Example 5

### Amide of adenosine 5′-triphosphate with Alexa 647

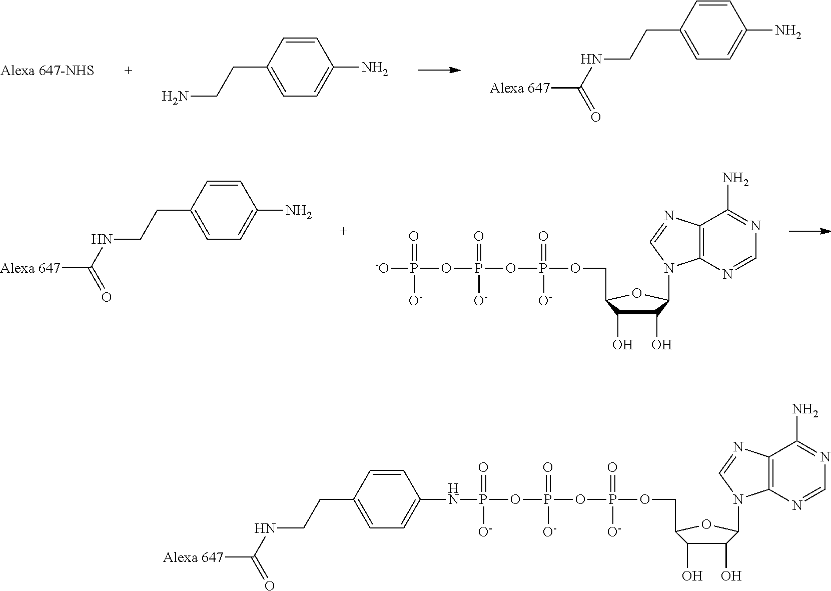

Alexa-647 as active ester (Molecular Probes; 1.0 mg) and 2-(4-aminophenyl)ethylamine (0.18 mg) were dissolved in the mixture of 1,4-dioxane (50 μL), water (20 μL) and 0.1M sodium bicarbonate (10 μL). The mixture was stirred overnight and the product was precipitated with acetone. The precipitate, adenosine-5′-triphosphate disodium salt (0.9 mg) and EDAC (0.6 mg) were dissolved in MES buffer (240 μL, 0.5 M, pH 5.5), and the mixture was stirred overnight at room temperature. The product was precipitated with acetone and was washed with the same solvent. The product was purified with HPLC and was analyzed with ESI-TOF mass spectrometry.

### Example 6

### Amide of adenosine 5′-triphosphate with {2,2′,2″,2′″-{[4′-(4′″-aminophenyl)-2,2′:6′,2″-terpyridine-6,6″-diyl]bis(methylenenitrilo)}tetrakis-acetate)}europium(III)

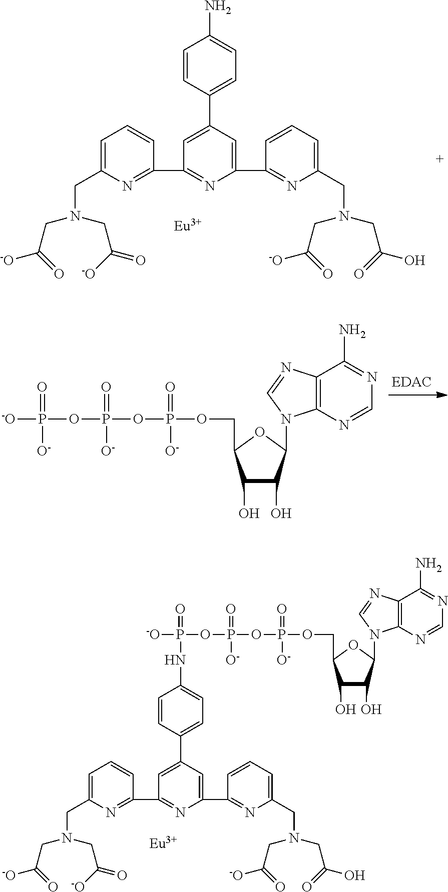

Adenosine 5′-triphosphate trisodium salt (2.1 mg) and {2,2′,2″,2′″-{[4′-(4′″-aminophenyl)-2,2′:6′,2″-terpyridine-6,6″-diyl]bis(methylenenitrilo)}tetrakis-(acetate)}europium(III) (3.3 mg) were dissolved in MES buffer, pH 5.5 (100 μL). EDAC (3.0 mg) was added and the reaction mixture was stirred overnight at RT. The product was precipitated with acetone (3 mL). The precipitation was washed with acetone. The product was purified with HPLC and was analyzed with ESI-TOF mass spectrometry.

### Example 7

### Amide of adenosine 5′-[β,γ-S]triphosphate with {2,2′,2″,2′″-{[4′-(4′″-aminophenyl)-2,2′:6′,2″-terpyridine-6,6″-diyl]bis(methylenenitrilo)}tetrakis-(acetate)}europium(III)

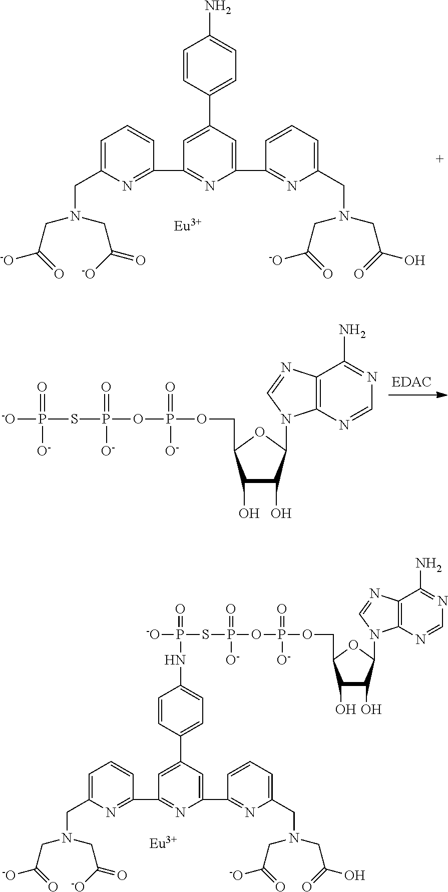

The title compound was synthesized according to the method disclosed in Example 2 but by using adenosine 5′-[β,γ-S]triphosphate as a starting material.

### Example 8

### Amide of adenosine 5′-[β,γ-imino]triphosphate with {2,2′,2″,2′″-{[4′-(4′″-aminophenyl)-2,2′: 6′,2″-terpyridine-6,6″-diyl]bis(methylenenitrilo)}-tetrakis(acetate)}europium(III)

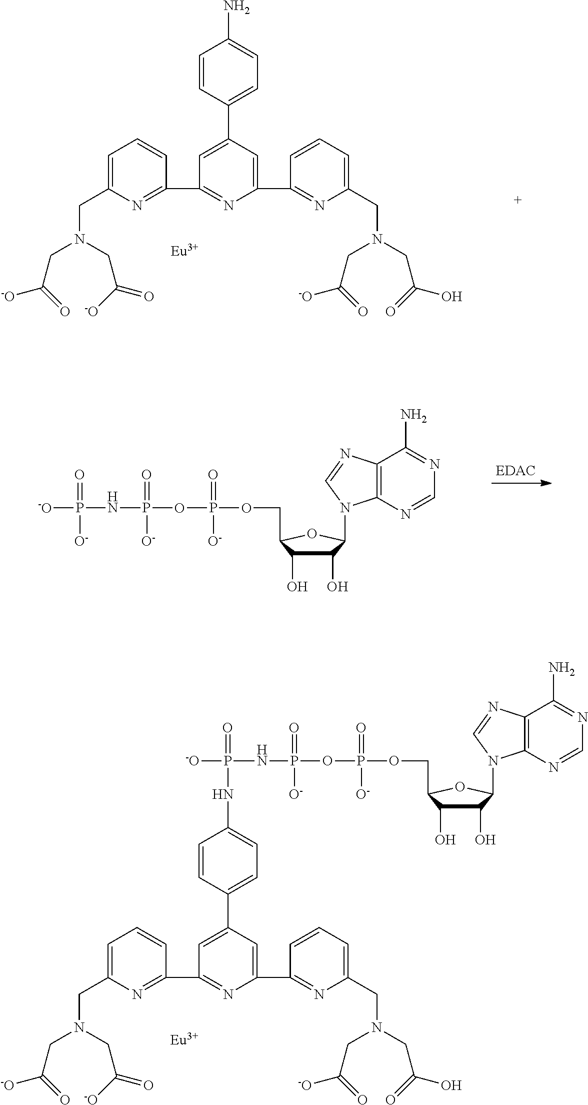

The title compound was synthesized according to the method disclosed in Example 2 but by using adenosine 5′-[β,γ-imino]triphosphate as a starting material.

### Example 9

### Labelling of non-hydrolysable adenosine-5′-triphosphate derivative in 2′-position

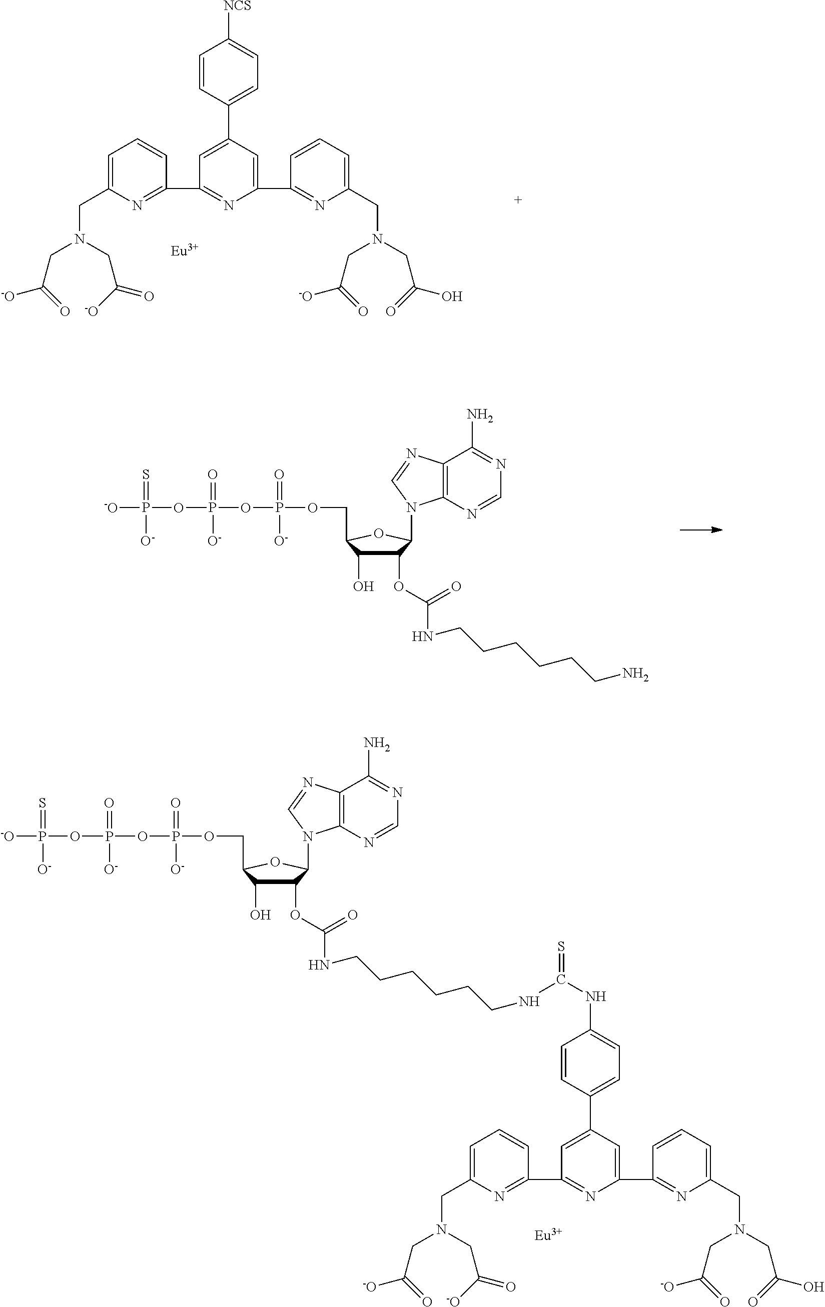

2′-(6-Aminohexylsemicarbazide)adenosine 5′-[γ-thio]-triphosphate and {2,2′,2″,2′″-{[4′-(4′″-isothiocyanatophenyl)-2,2′:6′,2″-terpyridine-6,6″-diyl]bis(methylenenitrilo)}tetrakis(acetate)}europium(III) were dissolved in the mixture of pyridine, triethylamine and water (9:1.5:0.1, v/v/v), and. the solution was stirred overnight at room temperature. The product was purified with HPLC.

### Example 10

### Labelling of non-hydrolysable adenosine-5′-triphosphate derivative in 8-position

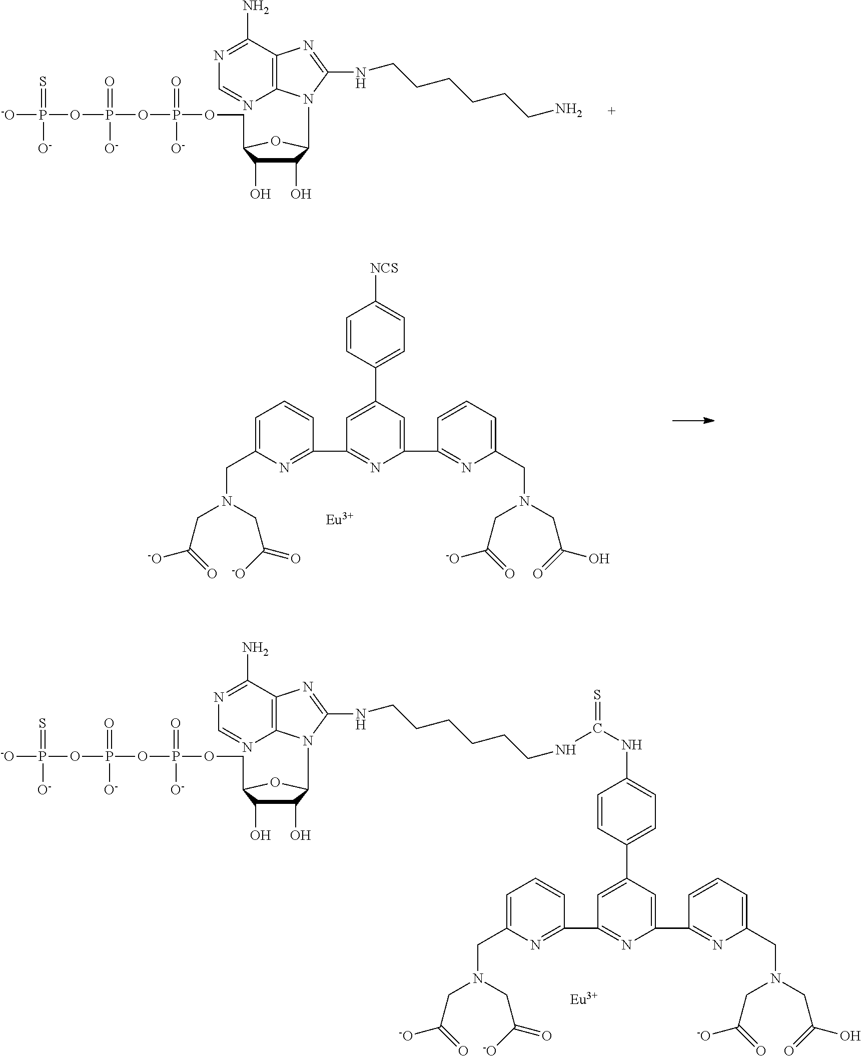

The synthesis was performed according to the method disclosed in Example 9 but by using 8-(6-aminohexyl)adenosine 5′-[γ-thio]-triphosphate as the starting material.

### Example 11

### Homogeneous Assays

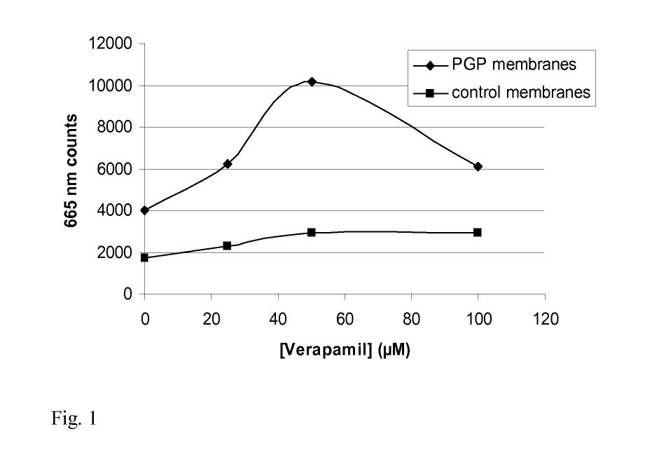

Eu-labeled ATP (donor, Example 1) and Alexa-647 labeled ATP (acceptor, Example 5) were used to measure the activation through energy transfer between these molecules bound to the adjacent NB sites of the same ABC transporter molecule. The assay was performed using Sf9 cell membrane preparations over-expressing ABC transporters MRP2 or PGP. The same cell line membranes transfected with same vector without transporter insert were used as controls. The membrane preparations (1 μg) in a MES buffer were incubated in lid covered 384-well microtitration plates (Wallac black plates or Wallac white Optiplates) at 37° C. for 5-20 min with varying concentrations of transporter specific substrates (probencid for MRP2 and verapamil for PGP) to get the efflux mechanisms activated. To diminish ATPase activity MgCl₂ was not included. After transporter activation, a reaction mixture containing 10 nM Eu-labeled ATP and 10 nM Alexa-647 labeled ATP were added, and the reaction mixture was incubated for further 2 hours. Duplicate reactions were performed using orthovanadate (Na₃VO₄, 1 mM) to measure the vanadate insensitive background. The reaction mixtures were measured directly without further separation by a time-resolved fluorometer (Victor 2) at 665 nm using 50 us delay and a 200 us counting window. To validate the eventual compound interference, measurements were performed also for the signal at 615 nm using the same time-window. These experiments were essential for the measurement of corrected energy-transfer signal. A dose-response curve with PGP transporter using verapamil as stimulating drug is given in FIG. 1.

It will be apparent for an expert skilled in the field that other embodiments exist and do not depart from the spirit of the invention. Thus, the described embodiments are illustrative and should not be construed as restrictive.

1. A homogenous proximity assay for ABC transporter activity, wherein detection of the ABC transporter activity is based on a signal between two labelled transporter binding molecules bound to ATP binding domains or sites adjacent to ATP binding domains.

2. The assay according to claim 1, wherein one or two of said transporter binding molecules are labelled ATP derivatives.

3. The assay according to claim 1, wherein one or two of said transporter binding molecules are selected from a group consisting of anti-transporter antibodies, lectins, oligopeptides, polypeptides, oligonucleotides, polynucleotides and anti-tag antibodies.

4. The assay according to any one of claim 1, wherein the signal detection is based on fluorescence energy transfer, fluorescence energy quenching, energy transfer between upconverted particles and fluorescent acceptors, fluorescence cross-correlation, luminescent oxygen channelling, or enzyme fragment complex formation upon proximity.

5. The assay according to claim 4, wherein the signal detection is based on time-resolved fluorescence energy transfer.

6. The assay according to claim 1, wherein the transported binding molecules are labelled with luminescent lanthanide(III) chelates, quantum dots, nanobeads, upconverting phosphors or organic dyes.

7. The assay according to claim 6, wherein the organic dye is selected form a group consisting of alexa dyes, cyanine dyes, dabcyl, dancyl, fluorescein, rhodamine, TAMRA and bodiby.

8. The assay according to claim 2, wherein one ATP is labelled with a luminescent lanthanide(III) chelate and one ATP is labelled with an organic dye.

9. The assay according to claim 2, wherein the ATP derivatives are stable towards nucleases.

10. The assay according to claim 2, wherein the labelled ATP is selected for the group consisting of

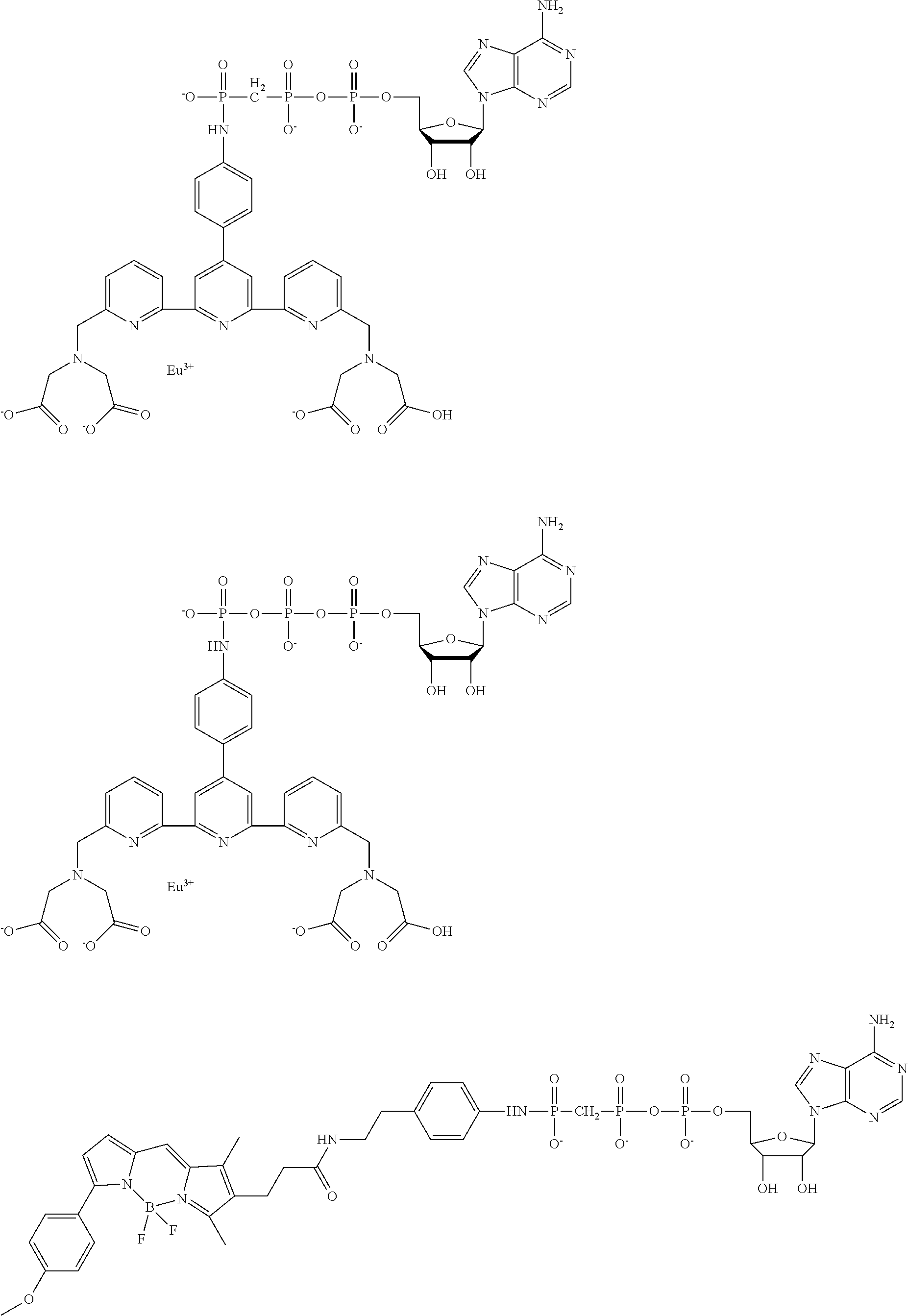

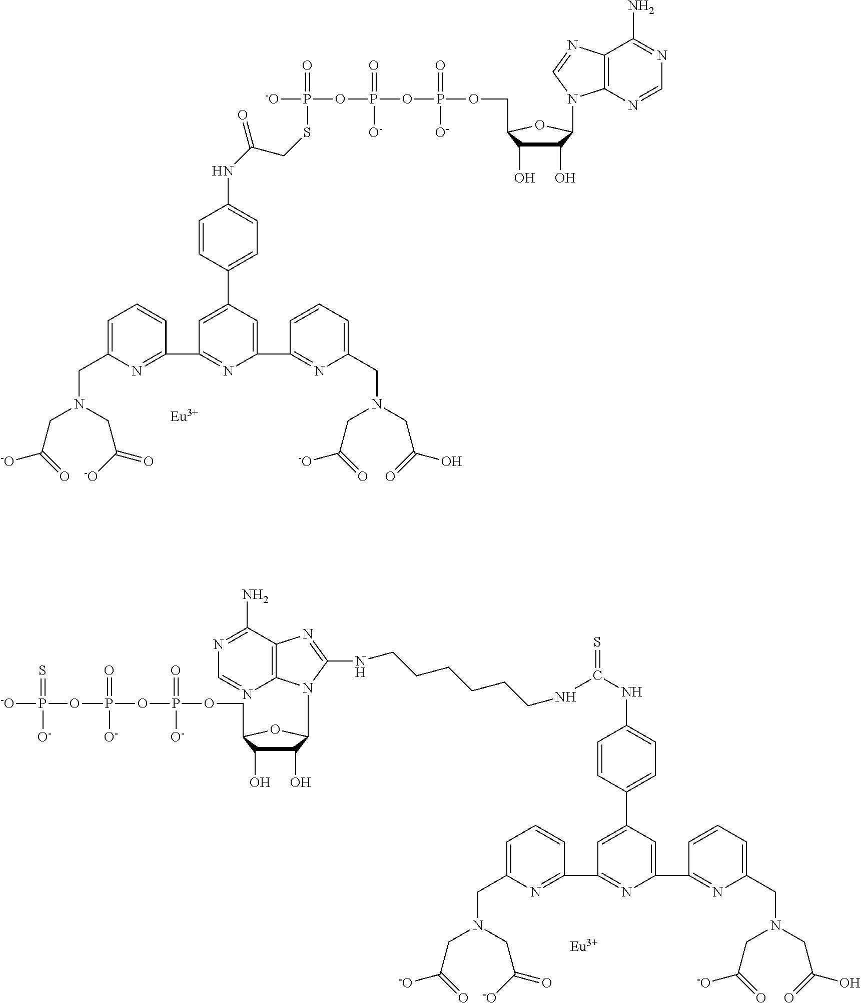

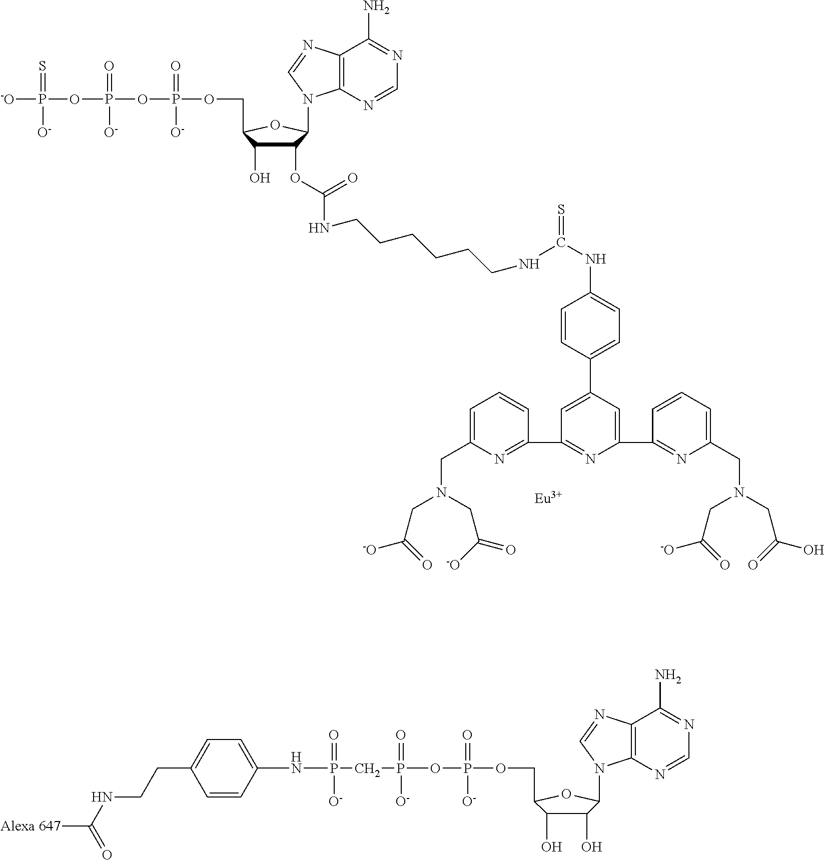

11. A method for measuring ABC transporter activity, comprising
incubating two labelled ABC transporter binding molecules with ABC transporter molecule, one of said transporter binding molecules being labelled with an energy donor and the other one with an energy acceptor, said ABC transporter binding molecules binding to ATP binding domains or sites adjacent to ATP binding domains of the said ABC transporter molecule; and
detecting the energy transfer signal between the two labelled ABC transporter binding molecules.
